(19) United States
(12) Patent Application Publication
(10) Pub. No.: US 2004/0152948 A1
(43) Pub. Date: Aug. 5, 2004

(54) AUXILIARY RING FOR GENITALS

(76) Inventor: Ee Do Kim, Anyang-Si (KR)

Correspondence Address: CANTOR COLBURN LLP, 55 Griffin Road South, Bloomfield, CT, 06002, US

(21) Appl. No.: 10/446,362

(22) Filed: May 28, 2003

(30) Foreign Application Priority Data
Feb. 4, 2003 (KR) 20-2003-0003217

(51) Int. Cl. A61F 5/00

(52) U.S. Cl. 600/38

(57) ABSTRACT

An auxiliary ring for genitals includes a rubber ring installed on the outer circumferential portion thereof, and filled with a liquid-phase material such as a bactericide or a lubricant. Thus, the liquid-phase material filled in the rubber ring is discharged during performance of a sexual act, and thus the skin of women can be protected from damage which may be caused by the auxiliary ring or from bacteria. Also, women who are in the menopause can enjoy the sexual lives without feeling pain. The genitals auxiliary ring which is a sexual organ assistance instrument, and is divided into a left ring and a right ring, in which respective one-side ends are hinged and the other-side ends can be detached from each other, includes a plurality of hooks which are extensively formed on the outer circumferential surfaces of the left ring and the right ring, and a rubber ring fitted into the hooks, and filled with a liquid-phase agent, in which at least one discharging hole is further formed on the outer circumferential surface of the rubber ring.

### BACKGROUND OF THE INVENTION

1. Field of the Invention

The present invention relates to an auxiliary ring which is worn around genitals in order to enhance vitality of the genitals which are contracted and expanded by a blood pressure of the genitals, and more particularly, to an auxiliary ring for genitals which includes hooks on the outer circumferential portion of the auxiliary ring, and a rubber ring fitted into the hooks, containing a liquid-phase material such as a bactericide or a lubricant, in which the liquid-phase material filled in the rubber ring is discharged during performance of a sexual act, and thus the skin of women can be protected from damage which may be caused by the auxiliary ring or from bacteria, and women who are in the menopause can enjoy the sexual lives without feeling pain.

2. Description of the Related Art

In general, erection of genitals occurs by a composite working of endocrine systems such as a nervous system, a blood system, and the hormone of a pituitary gland body. The male genitals have a special structure to respond to such a composite working of the endocrine systems. The male genitals are made of a sponge body including many blood tubes. When sexual stimulation is applied to the male genitals, the blood tubes in the male genitals are expanded and thus much blood enters the blood tubes. As a result, the male genitals become large by a blood pressure in the male genitals and get erected. In this case, the inside of the male genitals is filled with the blood and becomes hardened if the expansion of the male genitals reaches a high state of excitement.

The impotency means that the male genitals are not so sufficiently erected, and erection thereof is not so maintained for a sufficient time, as to perform a satisfactory sexual life. The factor of the impotency is divided into a mental factor and a physical factor. Of the two factors, the physical factor is caused by an abnormal artery which does not flow the blood smoothly, or an abnormal chemical reaction which does not erect the male genitals as desired. That is, since the blood tubes are not so sufficiently expanded that the male genitals cannot be filled with the blood.

Men who are weak in view of the sexual stamina use a sexual organ assistant agent to smooth the blood circulation of the genitals. In the past, an erection promotive agent in a gen form has been rubbed on a sensitive skin or an identical medicine has been taken to persons who have different physical features. As a result, the efficacy thereof becomes inconsistent, and taking the medicine which is not supported by a sufficient clinical demonstration may cause a harmful effect.

Also, an auxiliary ring in which jade emitting infra-red rays or permanent magnets generating magnetic forces are attached in order to reinforce stamina, is fitted into the genitals to thereby solve the above-described problems. In this case, the thickness of the genitals vary according to the erection degree of the genitals, which causes an inconvenience in wearing and a difficulty in use.

An example of the conventional auxiliary ring for genitals which was proposed in order to solve the above-described problems is disclosed in Korean Utility Model Registration No. 200624.

FIG. 1 is a perspective view illustrating a conventional ring for reinforcing a sexual stamina disclosed in Korean Utility Model Registration No. 200624. As show in FIG. 1, the conventional ring is worn around the male sexual organ, in which jade 3 and a permanent magnet 4 for emitting infra-red rays or magnetic forces, respectively are attached on the inner surfaces of annular metal rings 2 and 2′ which are opened to the left and right around a lower connection pin 1. In the case of the conventional ring, a tooth-shaped ratchet gear 5 is formed in a multi-stage in one end of the coupled portion of the metal rings 2 and 2′ harmless to the skin of the human body such as silver, and an opening and closing lever 7 is engaged and locked with the ratchet gear 5 by pressing a minute protrusion 6. Here, the ratchet gear 5 and the opening and closing lever 7 are elastically supported by a spring which is axially installed with a connection pin 8 in the other coupled portion of the metal rings 2 and 2′.

The conventional sexual stamina reinforcement ring having the above-described structure can be adjusted in size according to the erection degree of the male genitals, by using the ratchet gear 2 and 2′ and the opening and closing lever 7, to thus stimulate the sensitive skin tissue of the male genitals consistently to reinforce the sexual stamina.

Another example of the conventional ring for genitals, was proposed in Korean Utility Model Registration No. 298205 entitled a ring having an automatically adjustable inner diameter, for reinforcing a sexual stamina.

FIG. 2 is a perspective view showing a ring for enhancing capabilities of genitals disclosed in Korean Utility Model Registration No. 298205. A ring 11 is divided into left and right rings 12 and 13. The lower ends of the divided left and right rings 12 and 13 are combined by a coupling pin 23. Springs 15 and 17 are elastically supported to the coupling pin 23 so that the left and right rings 12 and 13 are folded. An adjustment unit 24 for automatically adjusting the inner diameter of the ring 11 is formed in the upper portions of the left and right rings 12 and 13. The adjustment unit 24 is formed as an arc-shaped tunnel from the end of the right ring 13 to a predetermined depth. First and second steps 19 and 20 are formed close to the inner surface of the right ring 13 in the inner circumferential surfaces of the tunnel. A throughhole 18 communicating with the inner surface of the right ring is formed in coupling holes 21, 22a, and 22b between the first and second steps 19 and 20. Also, an operating lever 25 is extended in the form of an arc so as to be inserted into the coupling holes from the end of the left ring 12 having a loop selectively caught by the first and second steps 19 and 20 in the coupling holes. Also, a button 26 engaged with the throughhole together with the springs releases the loop caught by the first and second steps with a pressure applied from the inner side of the ring.

Thus, the inner diameter of the ring can be automatically adjusted according to the erection degree of the genitals, to thereby provide an effect of further enhancing the blood circulation of the erected genitals.

However, in the case of the above-described two conventional sexual stamina auxiliary rings, the left and right rings are widened and narrowed according to the size of the erected male genitals, to thereby avoid the male genitals from being excessively pressed. However, when a man who wore the sexual stamina auxiliary rings performs a sexual act, the sexual organ of a woman who performs the sexual act together with him may be frequently damaged by the auxiliary rings.

Also, as the male genitals are erected earlier than the female genitals by using the conventional auxiliary rings, women may feel pain during performance of a sexual act. Since such a phenomenon becomes severe, an additional lubricant such as a gel-shaped agent is inclined to be frequently used.

In addition, the female genitals of the sensitive woman can be harmed by bacteria or microbes. However, since the conventional auxiliary rings purposed only the erection of the male genitals, the above-described conventional problems cannot be solved.

### SUMMARY OF THE INVENTION

To solve the above problems, it is an object of the present invention to provide an auxiliary ring for genitals including a rubber ring installed on the outer circumferential portion thereof, and filled with a liquid-phase material such as a bactericide or a lubricant, in which the liquid-phase material filled in the rubber ring is discharged during performance of a sexual act, and thus the skin of women can be protected from damage which may be caused by the auxiliary ring or from bacteria, and women who are in the menopause can enjoy the sexual lives without feeling pain.

To accomplish the above object of the present invention, there is provided an auxiliary ring for genitals comprising: a plurality of hooks which are extensively formed on the outer circumferential surfaces of a left ring and a right ring; and a rubber ring fitted into the hooks, and filled with a liquid-phase agent, in which at least one discharging hole is further formed on the outer circumferential surface of the rubber ring.

Also, the auxiliary ring further comprises a cover installed in the discharging hole formed in the outer circumferential surface of the rubber ring, for preventing the filled liquid-phase agent from being discharged during a non-use of the rubber ring.

the auxiliary ring for genitals according to the present invention provides effects that women who are in the menopause can feel less pain during performance of a sexual act, bacteria or microbes which may be infected to women can be prevented during performance of the sexual act, and the male genitals can smoothly erect, by the liquid-phase agent filled in the rubber ring provided in the outer circumferential surface of the ring.

### BRIEF DESCRIPTION OF THE DRAWINGS

The above and other objects and advantages of the present invention will become more apparent by describing the preferred embodiment thereof in more detail with reference to the accompanying drawings in which:

FIG. 1 is a perspective view illustrating a conventional ring for reinforcing a sexual stamina;

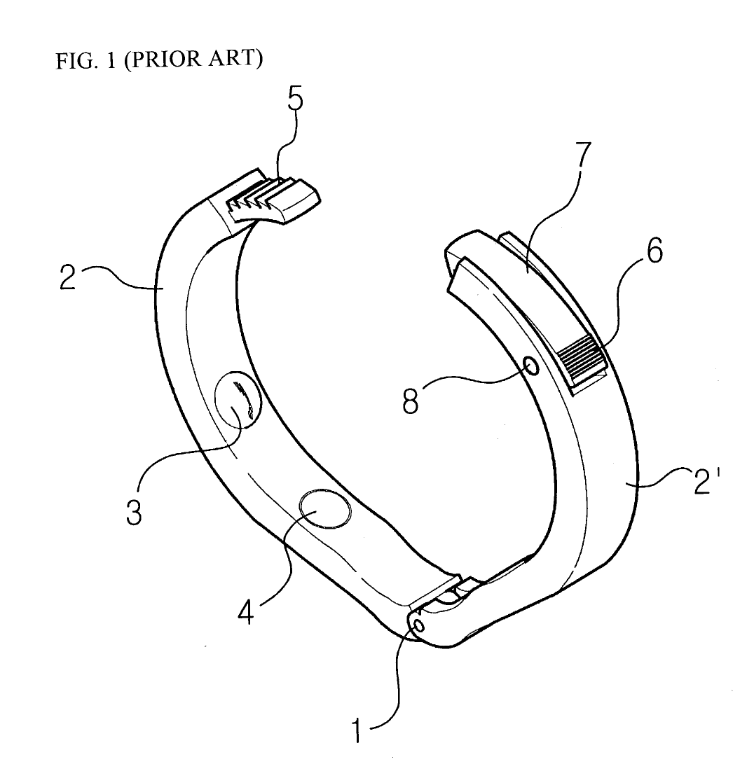

FIG. 2 is an exploded perspective view illustrating another conventional ring for reinforcing a sexual stamina;

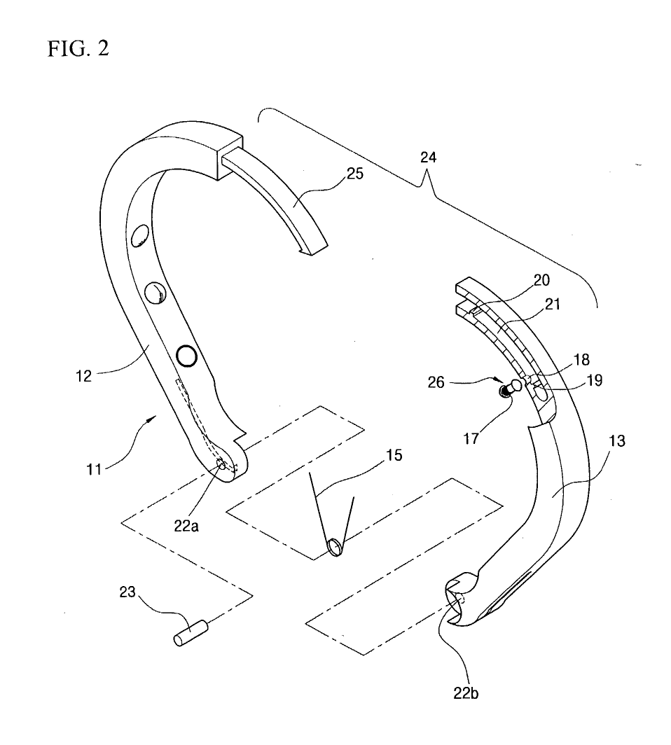

FIG. 3 is a perspective view illustrating an auxiliary ring for genitals according to the present invention;

FIG. 4 is a cross-sectional view of a line II-II of FIG. 3;

FIG. 5 is a front view illustrating the structure of the auxiliary ring for genitals according to the present invention; and

FIG. 6 is a cross-sectional view illustrating a rubber ring among the structural elements of the auxiliary ring according to another embodiment of the present invention.

### DETAILED DESCRIPTION OF THE INVENTION

The structure and function of the auxiliary ring for genitals according to the present invention will be described with reference to the accompanying drawings.

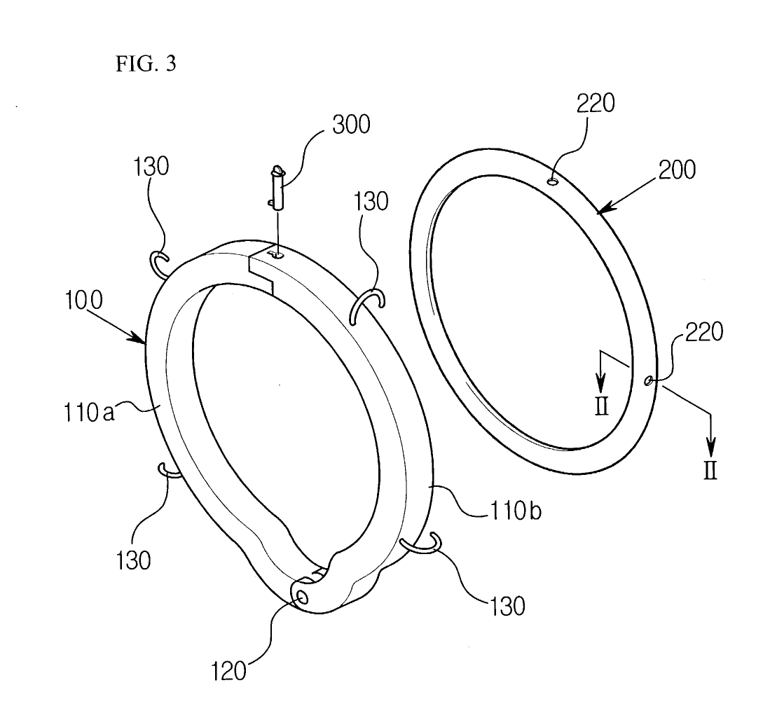

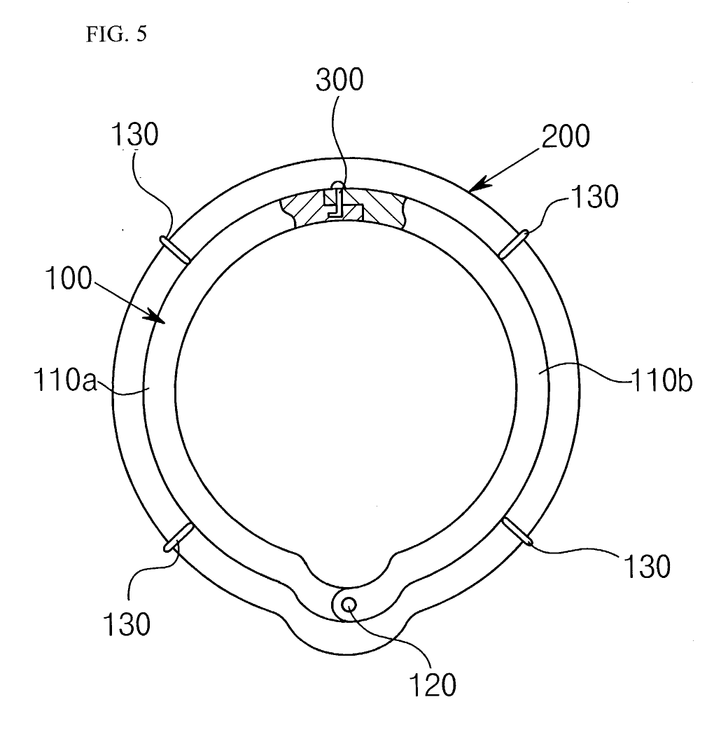

FIG. 3 is an exploded perspective view illustrating an auxiliary ring for genitals according to the present invention. FIG. 5 is a front view illustrating the structure of the auxiliary ring for genitals according to the present invention. As shown in FIGS. 3 and 5, the auxiliary ring 100 for genitals according to the present invention is divided into a left ring 110a and a right ring 110b. The left ring 110a and the right ring 110b are widened and narrowed by a hinge portion 120. Also, the auxiliary ring 100 further includes a plurality of hooks 130 on the circumferential surfaces of the left and right rings 110a and 110b, and a rubber ring 200 fitted into the hooks 130 formed on the circumferential surface of the auxiliary ring 100.

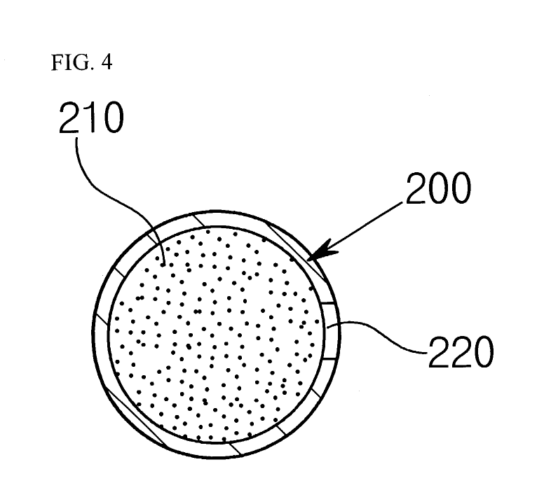

FIG. 4 is a cross-sectional view of a line II-II of FIG. 3. As shown in FIG. 4, the rubber ring 200 is filled with a liquid-phase agent 210, and is provided with at least one discharging hole 220 on the outer circumferential portion thereof. The left ring 110a and the right ring 110b are detachably formed on the other end-side portion thereof by a connection pin 300.

It is preferable that the liquid-phase agent filled in the rubber ring 200 is a lubricant containing a disinfection function for preventing the persons who perform a sexual act from being infected from bacteria.

When a man who suffers from impotency and is fitted with the auxiliary ring for genitals, perform a sexual act, the liquid-phase agent 210 in the rubber ring 200 provided around the auxiliary ring 100 is discharged through a discharging hole 220 on the rubber ring 200. Thus, a phenomenon that the old women who are not ready to perform a sexual act feel pain can be prevented in advance.

Also, since the rubber ring 200 can be purchased apiece, the auxiliary ring 100 can be continuously used after replacement of only the rubber ring 200. Thus, a burden of expenses becomes small.

It is preferable that the auxiliary ring 100 fitted with the rubber ring 200 and the hooks 130 extensively formed on the outer circumferential surface of the auxiliary ring 100 are made of an identical material. The material is not limited to a specific material, but it is preferably made of a rigid synthetic resin which does not harm the human body.

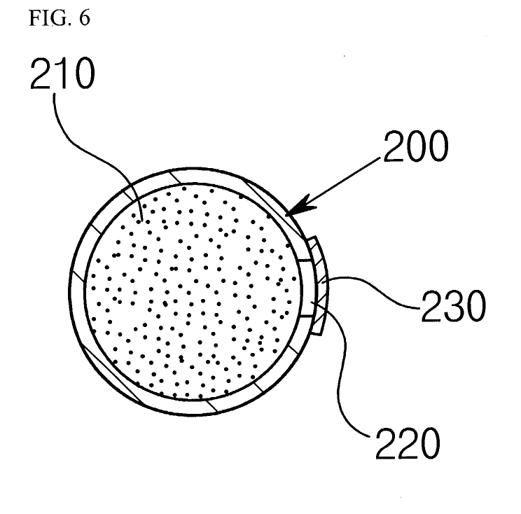

FIG. 6 is a cross-sectional view illustrating a rubber ring among the structural elements of the auxiliary ring according to another embodiment of the present invention. As shown, a cover 230 covering the openings on the outer circumferential surfaces of a plurality of discharging holes 220 formed on the outer circumferential surface of the rubber ring 200 is further provided.

The unused liquid-phase agent 210 filled in the rubber ring 200 can be prevented from being leaked out by the cover 230 in advance. It is preferable that the cover 230 is configured to be easily detached from the rubber ring 200.

As described above, the male genitals of men who suffer from impotency can be expedited to erect with the auxiliary ring for genitals according to the present inventions and a phenomenon that the old women who are not ready to perform a sexual act feel pain during a performance of a sexual act can be prevented in advance.

What is claimed is:

1. An auxiliary ring for genitals comprising:
a plurality of hooks which are extensively formed on the outer circumferential surfaces of a left ring and a right ring; and
a rubber ring fitted into the hooks, and filled with a liquid-phase agent, in which at least one discharging hole is further formed on the outer circumferential surface of the rubber ring.

2. The auxiliary ring of claim 1, further comprising a cover installed in the discharging hole formed in the outer circumferential surface of the rubber ring, for preventing the filled liquid-phase agent from being discharged during a non-use of the rubber ring.

3. The auxiliary ring of claim 1, wherein said liquid-phase agent filled in the rubber ring is a lubricant having a disinfection function.
